(19) United States
(12) Patent Application Publication
(10) Pub. No.: US 2004/0082823 A1
(43) Pub. Date: Apr. 29, 2004

(54) Method of hydrocarbon stabilization

(75) Inventor: Olga Tarasovna Kasaikina, Moscow (RU)

Correspondence Address: HOGAN & HARTSON LLP, IP GROUP, COLUMBIA SQUARE, 555 THIRTEENTH STREET, N.W., WASHINGTON, DC, 20004, US

(73) Assignee: NEUROK LLC

(21) Appl. No.: 10/279,011

(22) Filed: Oct. 24, 2002

(51) Int. Cl. C07C 7/20; C10L 1/00

(52) U.S. Cl. 585/1

(57) ABSTRACT

The present invention is directed to a method of providing enhanced molecular stability to hydrocarbons, such as the various oils, lubricants, fuel and rubbers typically used in internal combustion engines. The invention generally comprises inhibiting the liquid phase oxidation of hydrocarbons by atmospheric oxygen at elevated temperatures through the micro-addition of non-toxic of alkyl organic additives. The alkyl organic additives are anionic surface active agents (ASAAs) of the general formula R—OSO₃M or R—OP₃Z₂, where “M” is Na or K, where “Z” is Na, K or an alkyl group containing between ten and fourteen carbons and where “R” is an alkyl group consisting of between six and sixteen carbons. The additives are generally used in quantities from 0.01 to 10 mass percent, at temperatures between 70°-170° C. and in the presence of an aromatic solvent.

### BACKGROUND OF THE INVENTION

1. Field of the Invention

The present invention relates to a method of providing enhanced molecular stability to hydrocarbon compounds, including those used as components of oils, lubricants, fuel and rubbers commonly utilized in internal combustion engines, through the micro-addition of non-toxic antioxidants. The invention is applicable to the fuel, energy, oil-refining and chemical industries. By improving the stability of oils, lubricants, fuel and rubbers, the invention optimizes and prolongs the utilization of these materials, thus extending engine service lives and benefiting the environment.

2. Discussion of the Related Art

Oxidation of organic materials due to exposure to atmospheric oxygen results in the degradation of material quality and the loss of desired chemical properties. In lubricants and oils, for example, rancidity, stratification and sedimentation can occur. Similarly, premature fatigue of rubbers and polymers can occur due to oxidative aging. To address these and similar problems, manufacturers add agents, frequently toxic or environmentally damaging, to materials in order to retard the oxidation process. Examples of these agents include substituted phenols, aromatic amines and heterorganic compounds containing sulfur, metal complexes or phosphorous.

The oxidation process occurs according to a free radical mechanism in which an organic molecule reacts with oxygen to form free radicals (RH+O₂ →R*+HO₂*). The alkyl/aromatic radical reacts with oxygen to form an intermediate (R*+O₂→[RO—O*]). Subsequently, this intermediate reacts with another molecule of starting material to form a hydroperoxide and yet another free radical ([RO—O*]+RH→R*+RO—OH). Thus, the process is self-perpetuating.

Once formed, hydroperoxides can accelerate the rate of oxidation because they can easily decay into more free radicals (RO—OH→RO*+OH*). Hydroperoxide formation, therefore, contributes significantly to the self-accelerated nature of oxidative degradation, especially after a prolonged induction period (i.e. hydroperoxide formation and accumulation). To prevent oxidation, it is necessary to simultaneously decrease existing free radical content and proactively prevent hydroperoxide degradation into “new” free radicals.

Traditionally, additives—stabilizers, inhibitors, or antioxidants—are used to prevent oxidation. These additives prevent further free radical formation by interacting with existing free radicals and interrupting the self-perpetuating oxidation process. There are several well-known additive systems currently used industrially. Many of these additive systems utilize materials requiring special production facilities, such as phenolic compounds, derivatives of aromatic amines, and/or organometallic compounds. Furthermore, production of these additives results in the formation of poisonous byproducts (for example, the commercial synthesis of phenols often results in dioxin formation). Consequently, the production, transport and exploitation of traditional stabilizers often have undesirable environmental and/or economic effect.

One method of lubricant stabilization requires using synergistic mixtures of additives that both react with free radicals and destroy hydroperoxides without further free radical formation.

Another stabilization technique comprises the introduction of properly proportioned multi-functional compositions as additives. These additives help ensure proper dispersion, improve viscosity and decrease engine temperatures in addition to providing antioxidative, anticorrosive and antiwear properties. A common component of these additive mixtures is an organic salt of calcium or magnesium such as sulfonate, phenolate, salicylate or a combination thereof.

In Russian Federation Patent No. 2,161,179 (Dec. 27, 2000), substances like zinc dialkyldithiophosphate or dithiocarbamate, aromatic amines and their derivatives have been used as antioxidants. When sets of additives are used, different high-capacity oil mixtures can be composed according to a known formula. These compositions may be used as engine oils, hydraulic liquids and other machine oils. The efficiency of using additive composition is questionable, however, because these compositions typically comprises of a large number of constituents which makes its application difficult and its antioxidative action insufficient.

According to Japanese Patent No. 09-013058 (Jan. 14, 1997) anionic surface-active agents (ASAAs), such as sulfonates, sulfates or phosphates of alkali or alkali-earth metals or ammonium have been used as surfactants in emulsified water/heavy oil mixtures. Use of ASAAs in these applications provides stabilization by preventing stratification of the emulsified mixtures, but have no catalytic effect on hydroperoxides.

U.S. Pat. No. 4,151,100 (Apr. 24, 1979) discloses using 0.01-2.0 mass percent of cobalt thiobisalkylphenolate and their coordination compounds in conjunction with alcohols, alkylamines and arylamines as antioxidants to stabilize lubricants and plastics. For example, use of these additives at 175° C. with hexadecane has an induction period of up to 48 hours (where the induction period is the amount of time required to absorb 1 mole of oxygen per 1 kg of the sample).

These cobalt-based antioxidants can also be synergistically combined with other known additives such as N-phenyl-1-naphthyl-amine for enhanced or specialized use. Furthermore, the cobalt thiobisalkylphenolates, as well as their coordination compounds, demonstrate energy absorption and anti-sedimentation properties. These cobalt antioxidants, however, provide limited stabilization for engine oils, fuel and lubricants. Additionally, use of cobalt can be cost prohibitive.

### SUMMARY OF THE INVENTION

The present invention is directed to a method of providing enhanced molecular stability to various oils, lubricants, fuel and rubbers typically used in internal combustion engines. The invention generally comprises the inhibition of liquid phase oxidation of hydrocarbons by atmospheric oxygen at elevated temperatures through the micro-addition of non-toxic of alkyl organic additives. The method provides improved stabilization properties of various oils and organic substances and increases oxygen absorption times by up to 180 hours or more.

The alkyl additives are anionic surface active agents (ASAAs) of the general formula R—OSO₃M or R—OP₃Z₂, where “M” is Na or K, where “Z” is Na, K or an alkyl group containing between ten and fourteen carbons (C₁₀-C₁₄) and where “R” is an alkyl group consisting of between six and sixteen carbons (C₆-C₁₆). The additives are generally used in quantities of 0.01 to 10 mass percent (mass %). The oxidation prevention process is carried out at temperatures between 70°-170° C. in the presence of an aromatic solvent taken in the quantity defined by the formula: P=ASe^{-bAr}, where “P” is the mass percent of phenol or its derivatives, “A” is an empirical coefficient between 0.01 and 1.0 that characterizes the type of phenol compound and which accounts for its reactivity in the reactions of chain breakage and continuation, “S” is the mass percent of added alkyl additive, “e” is the base of natural logarithm, “b” is an empirical coefficient between 1.0 and 10.0 that characterizes the type of alkylaromatic hydrocarbons in the base oil and “Ar” the mass percent of alkylaromatic hydrocarbons in the base oil. Representative aromatic solvents include, for example, 2,6-Di-tert-butyl-4-methylphenol (BHT), hydroquinone, phenol, gossypol, natural phenolic acids, aminophenol, toluene, xylene, ethylbenzene and their alkyl substituted derivatives and may be used quantities from 0.0001 to 0.1 mass percent.

In view of the above art, one goal of the present invention is the development of a method for oil stabilization in the process of liquid phase oxidation by atmospheric oxygen in the presence of alkyl-containing additives. A further objective of the present invention is the identification of reduced or non-toxic, anionic surface-active agents and phenol compounds and mixtures thereof for use as antioxidants in various oils, lubricants, fuel and rubbers typically used in internal combustion engines.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### DETAILED DESCRIPTION OF THE PREFERRED EMBODIMENTS

The present invention is directed to a method of providing enhanced molecular stability to various oils, lubricants, fuel and rubbers typically used in internal combustion engines.

The method of oil stabilization proposed comprises the inhibition of liquid phase oxidation of hydrocarbons by atmospheric oxygen at elevated temperatures through the micro-addition of non-toxic alkyl organic additives. The alkyl additives are anionic surface active agents (ASAAs) of the general formula R—OSO₃M or R—OP₃Z₂, where “M” is Na or K, where “Z” is Na, K or an alkyl group containing between ten and fourteen carbons (C₁₀-C₁₄) and where “R” is an alkyl group containing of between six and sixteen carbons (C₆-C₁₆). The additives are generally used in quantities of 0.01 to 10 mass percent (mass %). The oxidation prevention process is carried out at temperatures between 70°-170° C. in the presence of an aromatic solvent taken in the quantity defined by the formula: P=ASe^{-bAr}, where “P” is the mass percent of phenol or its derivatives, “A” is an empirical coefficient between 0.01 and 1.0 that characterizes the type of phenol compound and which accounts for its reactivity in the reactions of chain breakage and continuation, “S” is the mass percent of added alkyl additive, “e” is the base of natural logarithm, “b” is an empirical coefficient between 1.0 and 10.0 that characterizes the type of alkylaromatic hydrocarbons in the base oil and “Ar” the mass percent of alkylaromatic hydrocarbons in the base oil. Representative aromatic solvents include, for example, 2,6-Di-tert-butyl-4-methylphenol (BHT), hydroquinone, phenol, gossypol, natural phenolic acids, aminophenol, toluene, xylene, ethylbenzene and their alkyl substituted derivatives and may be used in quantities from 0.0001 to 0.1 mass percent.

The formula, P=ASe^{-bAr}, is also applicable when preparing two-component additives for engine oils, fuels, lubricants, etc. The formula coefficient “A” accounts for variations in the inhibitory activity of the phenol component; “b” is based on the nature of the alkylaromatic hydrocarbons (“Ar”) in the material to be stabilized because the quantity of phenol in the oxidation product differs depending on the various alkylaromatic hydrocarbons. For example, using the same SDS additive and n-decane, a 10-fold deceleration of oxidation is observed with the addition of 10 mass percent of cumene while addition of 10 mass percent of ethylbenzene results in a 26-fold decrease in oxidation.

Coefficient Calculations

Applicable coefficients for the formula P=ASe^{-bAr}, as presented in Tables A and B, were derived from the relative reactivity of phenolic antioxidants according to the Handbook of Antioxidants by E. T. Denisov and T. I. Denisova (CRC Press, 2000).

TABLE A
Coefficient “A” for Select Phenolic Compounds

| Phenol Compound | Coefficient A |
|---|---|
| Phenol | 0.5 |
| BHT | 0.01 |
| 6-Hydroxydihydroquinoline or hydroquinone | 0.005 |
| 8-Hydroxydihydroquinoline, gossypol or 3,4- | 0.001 |
| dihydroxycinnamic acid (caffeic acid) | |

TABLE B
Coefficient b for select phenolic compounds

| Alkylaromatic Compound | Coefficient b |
|---|---|
| Ethylbenzene | 1 |
| Cumene | 0.5 |
| Toluene | 0.1 |
| Xylene | 0.2 |

### SPECIFIC EXAMPLES

General Experimental Conditions:

Substrate (20-40 ml) (e.g. ethylbenzene, n-decane, cumene, mixtures thereof or, isoparaffinic oil) was combined with approximately 0.23 g of sodium dodecylsulfate (SDS) and 0.04 g phenolic antioxidant in a bubble type glass vessel equipped with a reverse condenser cooled with water. The mixture was heated in a thermostat between 120°-150° C. and bubbled with air at 1.6l/h. Sample aliquots (0.1-0.5 ml) were taken once an hour for several hours to determine hydroperoxide concentration. Hydroperoxide concentration was determined iodometrically. The induction period was defined for each experiment as the amount of time required to reach either 10 or 20 mM concentration. Inhibition efficiency was evaluated as the oxidation induction period with inhibitor versus the induction period without an inhibitor. A longer induction period indicates greater oxidation inhibitor efficiency.

The results of laboratory tests, as detailed below, have demonstrated that the invention can enhance the stability of different oils and organic substances based on the increase induction periods. These increases, ranging from 48 to over 180 hours, indicate improved antioxidative properties of the materials treated. All figures are in mass percent unless otherwise noted.

### Specific Examples

### Example 1

### Ethylbenzene Oxidation (at 120° C.)

TABLE 1

| Additive | Induction Period* (hrs) |
|---|---|
| None | 1 |
| 2,6-Di-tert-butyl-4-methylphenol (BHT) | 8 |
| .01 mM = 0.0022% | |
| 2,6-Di-tert-butyl-4-methylphenol (BHT) | 40 |
| 1 mM = 0.022% | |
| SDS, 1 mM = 0.27% | >180 |
| SDS, 0.7 mM = 0.05% | >220 |

*Initial hydroperoxide concentration is less than 0.5 mM. Induction period is the time required to accumulate 20 mM of peroxides (i.e. 0.64% of peroxide oxygen)

### Example 2

### Dodecane Oxidation (at 140° C.)

TABLE 2

| | Induction |
|---|---|
| Additive | Period* (hrs) |
| None | 0.25 |
| 2,6-Di-tert-butyl4-methylphenol (BHT); 3.1 mM = 0.068% | 3.5 |
| SDS, 20 mM = 0.58% | 0.9 |
| SDS, 20 mM (0.58%) + 2,6-Di-tert-butyl4-methylphenol | >13 |
| (BHT); 3.1 mM (0.068%) | |

*Initial hydroperoxide concentration = 20 mM; Induction period is the time required to accumulate an additional 20 mM of peroxides.

### Example 3

### Cumene Oxidation (at 120° C.)

TABLE 3

| Additive | Induction Period* (hrs) |
|---|---|
| None | 0.9 |
| Bis-(2-ethylhexyl)-sulfosuccinate (AOT) (2%) | 0.3 |
| Phenol, 0.1% | 7 |
| AOT (2%) + phenol (0.1%) | 8 |
| Tridecylphosphate (1%) | >12 |
| SDS, (0.21%) | >45.5 |
| SDS, (0.054%) | >40 |

*Induction period is the time required to accumulate 10 mM of peroxides (0.032% of peroxide oxygen.

### Example 4

### Sunflower Seed Oil Oxidation (at 80° C.)

TABLE 4
(Sunflower Seed Oil Oxidation with no SDS)

| Sample | Peroxide Content (mM) |
|---|---|
| Base oil | 7.52 |
| Oil (12 hours in O₂ flow at 80 C.) | 51.7 |
| Oil (1 hour in N₂ flow at 80 C.) | 54.5 |
| Oil (2 hours in N₂ flow at 80 C.) | 61.1 |

TABLE 5
(Sunflower Seed Oil Oxidation with SDS)

| Sample | Peroxide Content (mM) |
|---|---|
| Oil + SDS* (12 hours in O₂ flow at 80 C.) | 14.3 |
| Oil + SDS* (1 hour in N₂ flow at 80 C.) | 28.2 |
| Oil + SDS* (2 hour in N₂ flow at 80 C.) | 24.4 |

*SDS concentration is 0.13% (4.45 mM).

### Example 5

### n-Decane Oxidation (at 150° C.)

TABLE 6

| | | | SAA | PhOH | Induction* |
|---|---|---|---|---|---|
| Exp. | Substrate | Additive | % | % | (hrs) |
| 1 | n-Decane | None | 0 | 0 | 0.5 |
| 2 | n-Decane | Phenol | 0 | 0.1 | 3 |
| 3 | n-Decane | BHT | 0 | 0.005 | 11.6 |
| 4 | n-Decane | 6-Hydroxy- | 0 | 0.008 | 8.5 |
| | | dihydro-quinoline | | | |
| 4a | n-Decane | 6-Hydroxy- | 0 | 0.016 | 15.5 |
| | | dihydro-quinoline | | | |
| 5 | n-Decane | 8-Hydroxytetra- | 0 | 0.004 | 15 |
| | | hydroquinoline | | | |
| 6 | n-Decane | SDS** | 0.1 | 0 | 0.5 |
| 7 | n-Decane | SDS, phenol | 0.1 | 0.1 | 11.5 |
| 8 | n-Decane | SDS, phenol | 0.05 | 0.1 | 5 |
| 9 | n-Decane | SDS, phenol | 0.1 | 0.03 | 6 |
| 10 | n-Decane | SDS, BHT | 0.1 | 0.0025 | 20 |
| 11 | n-Decane + | None | 0 | 0 | 0.35 |
| | Cumene (9:1) | | | | |
| 12 | n-Decane + | SDS | 0.1 | 0 | 3.5 |
| | Cumene (9:1) | | | | |
| 13 | n-Decane + | SDS, phenol | 0.1 | 0.1 | >50 |
| | Cumene (9:1) | | | | |
| 14 | n-Decane + | None | 0 | 0 | 0.5 |
| | Ethylbenzene | | | | |
| | (9:1) | | | | |
| 15 | n-Decane + | SDS | 0.1 | 0 | 13 |
| | Ethylbenzene | | | | |
| | (9:1) | | | | |
| 16 | n-Decane + | SDS | 0.1 | 0 | >30 |
| | Ethylbenzene | | | | |
| | (1:1) | | | | |
| 17 | n-Decane | H₃PO₄ | 0.1 | 0 | 0.5 |
| 18 | n-Decane + | H₃PO₄ | 0.1 | 0 | >35 |
| | Ethylbenzene | | | | |
| | (9:1) | | | | |
| 19 | n-Decane + | SDS | 0.1 | 0 | 4 |
| | Toluene (9:1) | | | | |
| 20 | n-Decane | Dihexadecylhydro | 0.1 | 0.1 | 7 |
| | | phosphate, phenol | | | |

*Induction period is the time required to accumulate 10 mM of peroxides (0.032% of peroxide oxygen).
**After 3 hours the settled steady-state hydroperoxide concentration is 0.06 M

### Example 6

### Isoparaffin Oil Oxidation (at 150° C.)

TABLE 7

| | | | |
|---|---|---|---|
| | | Additive | Induction* |
| Exp | Additive | Concentration % | (hrs) |

| | | | |
|---|---|---|---|
| 1 | None | — | 0.4 |
| 2 | 2,6-Di-tert-butyl4-methylphenol | 0.036 (1.62 mM) | 2.5 |
| | (BHT) | | |
| 3 | Diisooctyldiphenylamine | 0.04 (1.08 mM) | 2.5 |
| 4 | 6-Hydroxi-dihydroquinoline | 0.026 (1.36 mM) | 4.5 |
| 5 | 8-Hydroxi-dihydroquinoline | 0.03 (1.62 mM) | 10.4 |
| 6 | SDS + BHT (10:1) | 0.5 | 175 |

*Induction period is the time required for accumulation of 10 mM of peroxides (0.032% of peroxide oxygen.

It will be apparent to those skilled in the art that various modifications and variations can be made to the present invention without departing from its spirit or scope. Thus, it is intended that the present disclosure embrace all reasonable modifications and variations of this invention provided that they come within the scope of any claims and their equivalents.

What is claimed is:

1. A method of hydrocarbon stabilization comprising the steps of adding an additive of the formula R—OSO₃M to hydrocarbons in the presence of a solvent, wherein “M” is selected from the group consisting of sodium and potassium and wherein “R” is an alkyl group containing between six and sixteen carbons.

2. The method of claim 1 wherein said hydrocarbons are selected from the group consisting of ethylbenzene, n-decane, cumene, isoparaffinic oil, sunflower seed oil and mixtures thereof.

3. The method of claim 1 wherein said hydrocarbons are present in a lubricant and comprise more than ten percent of said lubricant.

4. The method of claim 1 wherein said addition of said additive occurs at a temperature between approximately 70° and approximately 170° C.

5. The method of claim 1 wherein the quantity of said additive added to said hydrocarbons is determined by the formula P=ASe^{-bAr,}
wherein “P” is the mass percent of said solvent;
wherein “A” is an empirical coefficient between approximately 0.01 and approximately 1.0;
wherein “S” is the mass percent of said additive;
wherein “e” is the base of natural logarithm;
wherein “b” is an empirical coefficient between approximately 1.0 and approximately 10.0;
wherein “Ar” is the mass percent of said hydrocarbons.

6. The method of claim 1 wherein the quantity of said additive added is between approximately 0.01 and approximately 10 mass percent of said hydrocarbons.

7. The method of claim 1 wherein said additive is an anionic surface-acting agent.

8. The method of claim 1 wherein said additive is selected from the group consisting of sodium dodecylsulfate, sodium decylsulfate and sodium tridecylsulfate.

9. The method of claim 1 wherein said solvent is an aromatic solvent.

10. The method of claim 1 wherein said solvent is selected from the group consisting of 2,6-Di-tert-butyl-4-methylphenol (BHT), hydroquinone, phenol, gossypol, natural phenolic acids, aminophenol, toluene, xylene, ethylbenzene and their alkyl substituted derivatives.

11. The method of claim 1 wherein said solvent is between approximately 0.0001 and approximately 0.1 mass percent of said hydrocarbon concentration.

12. The method of claim 10 wherein said solvent is between approximately 0.0001 and approximately 0.1 mass percent of said hydrocarbon concentration.

13. A method of hydrocarbon stabilization comprising the steps of adding an additive of the formula R—OPO₃Z₂ to hydrocarbons in the presence of a solvent, wherein “Z” is selected from the group consisting of sodium, potassium and alkyl groups containing between ten and fourteen carbons and wherein “R” is an alkyl group containing between six and sixteen carbons.

14. The method of claim 13 wherein said hydrocarbons are selected from the group consisting of ethylbenzene, n-decane, cumene, isoparaffinic oil, sunflower seed oil and mixtures thereof.

15. The method of claim 13 wherein said hydrocarbons are present in a lubricant and comprise more than ten percent of said lubricant.

16. The method of claim 13 wherein said addition of said additive occurs at a temperature between approximately 70° and approximately 170° C.

17. The method of claim 13 wherein the quantity of said additive added to said hydrocarbons is determined by the formula P=ASe^{-bAr},
wherein “P” is the mass percent of said solvent,
wherein “A” is an empirical coefficient between approximately 0.01 and approximately 1.0,
wherein “S” is the mass percent of said additive,
wherein “e” is the base of natural logarithm,
wherein “b” is an empirical coefficient between approximately 1.0 and approximately 10.0;
wherein “Ar” is the mass percent of said hydrocarbons.

18. The method of claim 13 wherein the quantity of additive added is between approximately 0.01 and approximately 10 mass percent of said hydrocarbons.

19. The method of claim 13 wherein said additive is an anionic surface-acting agent.

20. The method of claim 13 wherein said additive is selected from the group consisting of phosphoric acid, alkyl-substituted phosphoric acids, tridecylphosphate and dihexadecylhydrophosphate.

21. The method of claim 13 wherein said solvent is an aromatic solvent.

22. The method of claim 13 wherein said solvent is selected from the group consisting of 2,6-Di-tert-butyl-4-methylphenol (BHT), hydroquinone, phenol, gossypol, natural phenolic acids, aminophenol, toluene, xylene, ethylbenzene and their substituted derivatives.

23. The method of claim 13 wherein said solvent is between approximately 0.0001 and approximately 0.1 mass percent of said hydrocarbon concentration.

24. The method of claim 22 wherein said solvent is between approximately 0.0001 and approximately 0.1 mass percent of said hydrocarbon concentration.

25. A method of hydrocarbon stabilization comprising the steps of adding a mixture of additives of the formula R—OSO₃M and R—OPO₃Z₂ to hydrocarbons in the presence of a solvent, wherein “M” is selected from the group consisting of sodium and potassium, wherein “Z” is selected from the group consisting of sodium, potassium and alkl groups containing between ten and fourteen carbons and wherein “R” is an alkyl group containing between six and sixteen carbons.

26. The method of claim 25 wherein said mixture of additives comprises between approximately 0.01 and approximately 99.99 mass percent of said additive of the formula R—OSO₃M, wherein “M” is selected from the group consisting of sodium and potassium and “R” is an alkyl group containing between six and sixteen carbons, and between approximately 0.01 and approximately 99.99 mass percent of said additive of the formula R—OPO₃Z₂, wherein “Z” is selected from the group consisting of sodium, potassium and alkyl groups containing between ten and fourteen carbons and wherein “R” is an alkyl group containing between six and sixteen carbons.

27. The method of claim 25 wherein said hydrocarbon is selected from the group consisting of ethylbenzene, n-decane, cumene, isoparaffinic oil, sunflower seed oil and mixtures thereof.

28. The method of claim 25 wherein said hydrocarbons are present in a lubricant and comprise more than ten percent of said lubricant.

29. The method of claim 25 wherein said addition of said mixture of additives occurs at a temperature between approximately 70° and approximately 170° C.

30. The method of claim 25 wherein the quantity of said mixture of additives added to said hydrocarbons is determined by the formula P=ASe^{-bAr},
wherein “P” is the mass percent of said solvent,
wherein “A” is an empirical coefficient between approximately 0.01 and approximately 1.0,
wherein “S” is the mass percent of said mixture of additives,
wherein “e” is the base of natural logarithm,
wherein “b” is an empirical coefficient between approximately 1.0 and approximately 10.0;
wherein “Ar” is the mass percent of said hydrocarbons.

31. The method of claim 25 wherein the quantity of said mixture of additives added is between approximately 0.01 and approximately 10 mass percent of said hydrocarbons.

32. The method of claim 25 wherein said mixture of additives is a mixture of anionic surface-acting agents.

33. The method of claim 25 wherein said additive of the formula R-OSO₃M is selected from the group consisting of sodium dodecylsulfate, sodium decylsulfate and sodium tridecylsulfate.

34. The method of claim 25 wherein said additive of the formula R-OPO₃Z₂ is selected from the group consisting of phosphoric acid, alkyl-substituted phosphoric acids, tridecylphosphate and dihexadecylhydrophosphate.

35. The method of claim 25 wherein said mixture of additives is comprised of additives selected from the group consisting of sodium dodecylsulfate, sodium decylsulfate, sodium tridecylsulfate, phosphoric acid, alkyl-substituted phosphoric acids, tridecylphosphate and dihexadecylhydrophosphate.

36. The method of claim 25 wherein said solvent is an aromatic solvent.

37. The method of claim 25 wherein said solvent is selected from the group consisting of 2,6-Di-tert-butyl-4-methylphenol (BHT), hydroquinone, phenol, gossypol, natural phenolic acids, aminophenol, toluene, xylene, ethylbenzene and their substituted derivatives.

38. The method of claim 25 wherein said solvent is between approximately 0.0001 and approximately 0.1 mass percent of hydrocarbon concentration.

39. The method of claim 37 wherein said solvent is between approximately 0.0001 and approximately 0.1 mass percent of hydrocarbon concentration.
